(19) United States
(12) Patent Application Publication
(10) Pub. No.: US 2008/0108864 A1
(43) Pub. Date: May 8, 2008

(54) Penis retractor for use in surgical or other medical procedures

(76) Inventors: Barbara Girgen, Salt Lake City, UT (US); Violet Joyce Schafer, Wildomar, CA (US)

Correspondence Address: Roy A. Ekstrand, 3158 Redhill, Ste 150, Costa Mesa, CA, 92626, US

(21) Appl. No.: 11/222,606

(22) Filed: Sep. 9, 2005

Related U.S. Application Data
(60) Provisional application No. 60/608,219, filed on Sep. 9, 2004.

(51) Int. Cl. A61F 5/00

(52) U.S. Cl. 600/41

(57) ABSTRACT

A penis retractor for use in retracting a patient's penis from an area of the patient's anatomy includes a tether forming a penis engaging loop together with one or more attachment pads which are secured to the patient's upper abdomen. In some embodiments, a cooperating abdominal belt encircles the patient's torso and provides attachment for the attachment pad or pads of the retractor.

### CROSS-REFERENCE TO RELATED APPLICATIONS

This application claims the benefit of and priority under 35 USC 119(e) of U.S. Provisional patent application No. 60/608,219 entitled G AND S PENIS RETRACTOR filed Sep. 9, 2004. The disclosure of which is incorporated herein by reference.

### FIELD OF THE INVENTION

This invention relates generally to surgical and medical examination procedures and particularly to apparatus for preparing the examination or surgical area for a medical procedure.

### BACKGROUND OF THE INVENTION

In certain medical procedures such as surgery or examination or treatment undertaken in the male groining or genital area, the patient's penis must be retracted or restrained to avoid undue interference in the procedure or contamination of the area of interest. For example, surgical procedures performed in the scrotum area, such as vasectomies, require that the patient's penis be restrained against the patient's lower abdomen.

For the most part, this restraint is provided by simply taping the penis against the frontal abdomen wall. Other “makeshift” methods of penis restraint are also employed by medical practitioners.

As can be readily understood, these methods of restraint such as taping or the like are often extremely uncomfortable for the patient. In their understandable zeal to ensure that the patient's penis does not become loose and intrude into the surgical or examination area, practitioner's often “over-restrain” the penis.

While the various methods heretofore employed by medical practitioners in restraining the patient's penis have for the most part proven adequate, the discomfort and sometimes painful consequences to the patient of such restraints create a need in the art for a system and apparatus for penis retraction or restraint which is simultaneously comfortable for the patient while being secure and reliable for medical practitioners.

### SUMMARY OF THE INVENTION

Accordingly, it is a general object of the present invention to provide an improved penis retractor or restraint for use in surgical or other medical procedures. It is a more particular object of the present invention to provide an improved penis retractor or restraint which is secure. It is a still further object of the present invention to provide a secure penis retractor for use in surgical or other medical procedures which avoids undue stress or discomfort upon the patient.

In accordance with the present invention, there is provided a penis retractor used in restraining the penis of a patient during surgical, examination or treatment procedures, the penis retractor comprising: an elongated flexible tether having first and second ends and a loop formed therein; at least one attachment pad joined to the first end; and means for adjusting the loop to encircle a portion of a patient's penis, the tether being drawn to retract and position a patient's penis and the attachment pad being securable to a patient's abdomen to maintain the position.

### BRIEF DESCRIPTION OF THE DRAWINGS

The features of the present invention, which are believed to be novel, are set forth with particularity in the appended claims. The invention, together with further objects and advantages thereof, may best be understood by reference to the following description taken in conjunction with the accompanying drawings, in the several figures of which like reference numerals identify like elements and in which:

FIG. 1 sets forth a front perspective view of a penis retractor constructed in accordance with the present invention;

FIG. 2 sets forth a partial perspective view of the penis retractor of FIG. 1 in a typical use upon a male patient;

FIG. 3 sets forth a front perspective view of the penis retractor of FIG. 1 utilizing a cooperating abdominal belt for attachment;

FIG. 4 sets forth a perspective view of an alternate embodiment of the present invention penis retractor having duel attachment points;

FIG. 5 sets forth a partial perspective view of the dual attachment point penis retractor of FIG. 4 in a typical use on a male patient;

FIG. 6 sets forth a front perspective view of the penis retractor of FIG. 4 in its typical use;

FIG. 7 sets forth a front view of a still further alternate embodiment of the present invention penis retractor;

FIG. 8 sets forth a partial front perspective view of the penis retractor of FIG. 7 in a typical use upon a male patient;

FIG. 9 sets forth a front perspective view of the embodiment of FIG. 7 used upon a patient without an abdominal belt;

FIG. 10 sets forth a partial perspective view of the embodiment of FIG. 7 in a typical use upon a male patient;

FIG. 11 sets forth a perspective view of a cooperating belt utilized in combination with the present invention penis retractor having hook and loop fabric attachment apparatus;

FIG. 12 sets forth a further embodiment of the cooperating belt having button and button hole attachment apparatus together with front buttons for retractor attachment;

FIG. 13 sets forth a cooperating belt for use with the present invention penis retractor having button hole and button attachment apparatus;

FIG. 14 sets forth a cooperating abdominal belt having hook and anchor tab attachment apparatus;

FIG. 15 sets forth a cooperating abdominal belt generally similar to that shown in FIG. 12 which utilizes a single front abdominal anchor button;

FIG. 16 sets forth a partial perspective view of an attachment pad utilized in an alternate embodiment of the present invention penis retractor which provides a button hole within the pad.

### DESCRIPTION OF THE PREFERRED EMBODIMENTS OF THE INVENTION

FIG. 1 sets forth a perspective view of a penis retractor constructed in accordance with the present invention and generally referenced by numeral 10. Retractor 10 includes an elongated preferably flexible tether 11 having ends 12 and 13. Tether 11 is tied to form a slip knot 14 and define a loop portion 15. End 15 of tether 11 is joined to an attachment pad 16 by conventional fabrication means which may include adhesive attachment or like. Attachment pad 16 is preferably formed to include an adhesive surface on the underside thereof which provides attachment to the skin of a patient or a cooperating abdominal belt secured to the users abdomen. Tether 11 is preferably formed of a flexible material which may for example include a suitable cord or suitable filament. Alternatively, tether 11 may be fabricated of a tubular material such as surgical tubing or the like. The important aspect in considering the fabrication of tether 11 is found in selecting a material which is suitably strong for the needs of retractor 10 while being sufficiently flexible to ensure comfortable tying of the patient penis as well as ease of attachment and so on.

FIG. 2 sets forth a partial perspective view of a typical patient 20 positioned for a surgical or examination procedure in an area 22 which, due to its location, raises the need to retract the patient's penis. Thus, the patient is positioned in FIG. 2 having penis 23 retracted by retractor 10 in a typical application of the present invention. Penis 23 includes a scrotum area 26, a penal shaft 25 and a head 24. In the procedure shown in FIG. 2, the procedure or surgery to be undertaken requires that penis 23 be retracted upwardly toward abdominal wall 21 of patient 20. Accordingly, penis retractor 10 is positioned such that tether 11 having slip knot 14 is secured to penis shaft 25 beneath head 24 by loop 15. End 12 is drawn to provide sufficient engagement of loop 15 upon penis 23. Thereafter, end 13 of tether 11 is drawn upwardly until a sufficient restraining force is applied to penis 23 after which attachment pad 16 is pressed against the outer skin of abdomen 21.

It will be apparent to those skilled in the art that the use of the present invention penis retractor positions penis 23 in an out of the way and positively restrained position to facilitate surgical procedures or examination by practitioners within the general area indicated as area 22. It will be equally apparent to those skilled in the art that retractor 10 operates with a maximum of security while simultaneously ensuring a maximum of comfort for the patient. It will also be noted that the engagement of penis 23 by loop 15 of tether 11 ensures that a secure attachment is formed without the negative effects of utilizing tape or other materials as are presently employed by practitioners in the medical arts.

FIG. 3 sets forth a partial front perspective view of penis retractor 10 utilizing a cooperating abdominal belt 30. Thus, patient 20 having abdominal wall 21 is positioned generally as described above such that penis 23 is drawn upwardly against abdominal wall 21 to expose an area 22 of interest to the practitioners. In the position shown in FIG. 3, penis 23 is positioned such that scrotum 26 is relaxed while penal shaft 25 and head 24 are drawn upwardly. It will be noted that retractor 10 having tether 11 is engaged upon penis 23 such that loop 15 of tether 11 is positioned behind head 24 and is secured by drawing end 12 of tether 11 through slip knot 14.

In the embodiment shown in FIG. 3, cooperating abdominal belt 30 which may be fabricated in any of the various fabrications shown and described below is positioned upon a convenient portion of abdomen 21 of patient 20. Thereafter, attachment pad 16 bearing end 13 of tether 11 pressed against the outer surface of abdominal belt 30 completing the attachment of retractor 10. Once again it will be noted that the use of loop 15 to engage penis 23 provides a minimum of discomfort to the patient while securing the penis in a reliable securement.

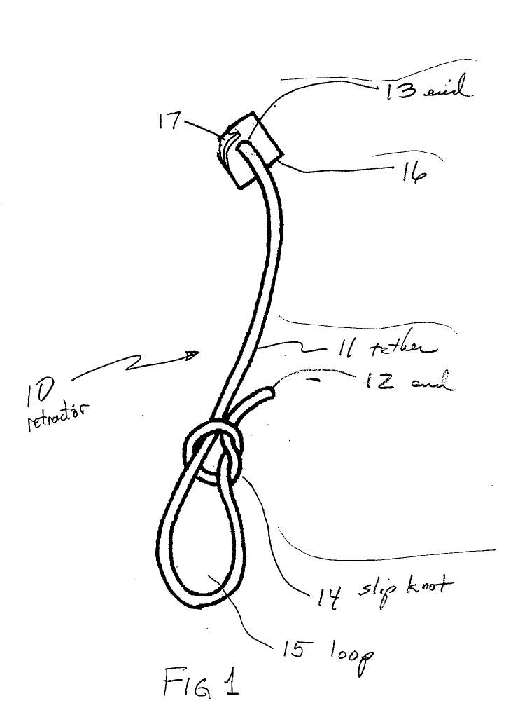

FIG. 4 sets forth a perspective view of an alternate embodiment of the present invention penis retractor generally referenced by numeral 40. Retractor 40 includes an elongated tether 41 fabricated of a flexible sufficiently strong material as mentioned above for tether 11. Tether 41 includes ends 42 and 43 secured to respective attachment pads 47 and 48 by conventional attachment (not shown). Tether 41 further forms a loop 44 and supports a clamp 45. Clamp 45 may be fabricated in accordance with conventional fabrication techniques with its essential feature being the ability to secure the underlying portions of tether 41 in a fixed attachment once loop 44 has been properly sized as described below. Alternately, clamp 45 may be replaced by other types of clamps such as a clamp 46 which generally resembles an “alligator clamp”. It will be apparent to those skilled in the art that a variety of conventional clamps may be utilized to secure tether 41 so as to form loop 44 without departing from the spirit and scope of the present invention. For example, within typical medical apparatus, a variety of devices are utilized in clamping tubing or supporting cords or filaments within the commonly available surgical apparatus.

FIG. 5 sets forth a partial perspective view of a patient 20 having a penis retractor 40 secured thereto in preparation for a surgical or examination procedure. As described above, patient 20 has an abdominal wall 21 and an area 22 which is the area of interest to the medical examiner and which generally covers the lower groining area of the patient. As described above, penis retractor 40 includes a tether 41 having ends 42 and 43 supporting attachment pads 47 and 48 together with a clamp 45 which forms tether 41 into a loop 44.

In the treatment position shown in FIG. 5, penis 23 of patient 20 is positioned such that scrotum 26 extends downwardly while shaft 25 and head 24 extend upwardly. Penis retractor 40 is secured to penis 23 such that loop 44 is positioned upon penal shaft 25 beneath head 24. Thereafter, clamp 45 and tether 41 are positioned to correctly size loop 44 and sufficiently engage penis 23 for retraction. An abdominal belt 50 is positioned upon abdomen 21 of patient 20 and encircles the patient's torso. Finally, attachment pads 47 and 48 of retractor 40 are drawn upwardly to provide sufficient retracting force upon penis 23 after which pads 47 and 48 are pressed down upon the outer surface of abdominal belt 50. In this manner, the retraction of penis 23 is completed. It will be noted that retractor 40 enjoys the advantage of having two portions of tether 41 extending outwardly in an angular manner from loop 15 and clamp 45. This provides additional side to side retention of penis 23 and is some instances advantageous in the event for example that the patient must be rotated or moved during surgical or examination procedures.

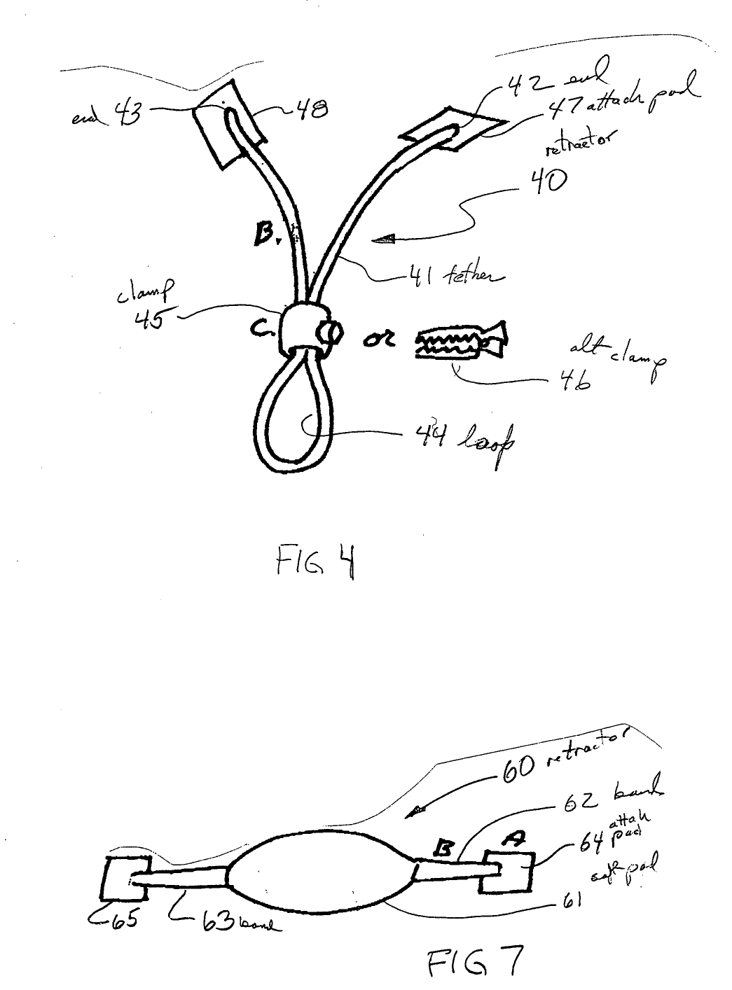

FIG. 6 sets forth a front perspective view of penis retractor 40 situated upon a patient 20 without the utilization of abdominal belt 50. In this case, penis retractor 40 functions in the same manner as described above in FIG. 5 with the difference being found in the application of attachment pads 47 and 48 directly upon the skin of abdomen 21 of patient 20. In addition, FIG. 6 provides a better illustration of the benefits of the angled portions of tether 41 extending upwardly and outwardly from clamp 45.

FIG. 7 sets forth a front view of a still further alternate embodiment of the present invention penis retractor generally referenced by numeral 60. Retractor 60 includes a central generally oval soft pad 61 together with a pair of outwardly extending flexible bands 62 and 63. Bands 62 and 63 are further joined to a pair of attachment pads 64 and 65 which may for example include an attachment adhesive on the underside thereof. It will be understood that the adhesive layer utilized on the undersides of attachment pads such as attachment pads 64 and 65 is employed in accordance with adhesive layer 17 shown in FIG. 1 of attachment pad 16. Soft pad 61 is preferably formed of a soft flexible material such as cotton cloth or elastic plastic or the like. Correspondingly, bands 62 and 63 are preferably formed of a flexible material such as plastic or the like having sufficient strength to maintain the patient's penis in the attachment shown in FIG. 8. Thus, bands 62 and 63 may for example be somewhat elastic plastic material or the like.

FIG. 8 sets forth a partial perspective view of a patient 20 having abdominal wall 21 positioned for a surgical or examination procedure within lower groining area 22. As described above, it is desired in this surgical or examination procedure to position penis 23 of patient 20 such that scrotum 26 is raised somewhat while penal shaft 25 having head 24 is drawn upwardly against abdomen 21.

Accordingly, an abdominal belt 50 is secured to patient 20 as described above and receives penal retractor 60. As may be appreciated, soft pad 61 is positioned upon penal shaft 25 beneath head 24 after which bands 62 and 63 are drawn outwardly with sufficient force to captivate penal shaft 25. Thereafter, attachment pads 64 and 65 are pressed against abdominal belt 50 to complete the retraction of penis 23.

FIG. 9 sets forth a partial perspective view substantially identical to the view shown in FIG. 8 in which abdominal belt 50 is not utilized. Thus, as described above, patient 20 is positioned such that area 22 is exposed and scrotum 26 and penal shaft 25 of penis 23 are drawn upwardly upon abdomen 21. As is also described above, penis retractor 60 is positioned such that pad 61 is pressed against penal shaft 25 beneath head 24 and secured in place by attachment pads 64 and 65. Unlike the utilization of retractor 60 shown in FIG. 8 however, pads 64 and 65 are pressed directly against the skin of abdomen 21 without utilizing abdominal belt 50.

FIG. 10 sets forth a partial side perspective view of a patient 20 having penis retractor 60 secured thereto in the manner shown in FIG. 8. As described above in FIG. 8, patient 20 having abdominal wall 21 and penis 23 is positioned to expose area 22. Thus, penal shaft 25 is oriented upwardly to slightly raise scrotum 26 while retractor 60 secures penis 23 in its retracted position utilizing cooperating abdominal belt 50. As is also described above, retractor 60 includes a soft pad 61 which captivates the upper portion of penal shaft 25 and secures it against belt 50 utilizing attachment pads 64 and 65 (pad 64 shown in FIG. 8).

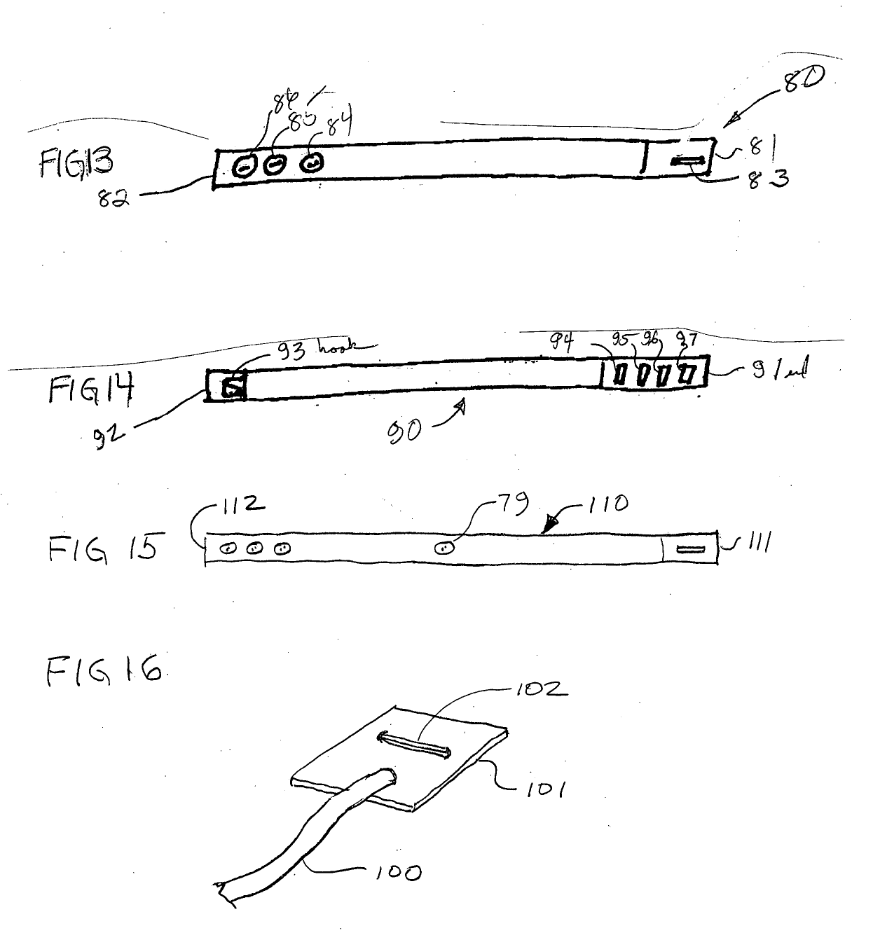

FIG. 11 sets forth a perspective view of a cooperating abdominal belt utilized in combination with the present invention penis retractor and generally referenced by numeral 30. Abdominal belt 30 may be fabricated of virtually any suitable material having sufficient strength and flexibility such as cloth or the like and is preferably formed to define a generally flat or ribbon like contour. However, it will be apparent to those skilled in the art that a great variety of shapes and cross sections may be utilized in forming an abdominal belt suitable for cooperating with the present invention penis retractor without departing from the spirit and scope of the present invention.

More specifically, abdominal belt 30 includes end portions 31 and 32. End 32 supports a first attachment pad 33 while end 31 supports a cooperating attachment pad 34. While a variety of attachments may be utilized such as adhesive or the like. It has been found preferable to utilize cooperating attachment pads of the type generally referred to as hook and loop fabric attachments. It will be important to note that attachment pads 33 and 34 are sufficient in span to provide size adjustment of abdominal belt 30 to accommodate different girths of patients.

FIG. 12 sets forth a further embodiment of the abdominal belt utilized in cooperation with the present invention penis retractor generally referenced by numeral 70. Once again belt 70 may be fabricated of a variety of materials as mentioned above having a sufficient strength and flexibility. Belt 70 has ends 71 and 72 supporting a button hole 73 and a plurality of buttons 74, 75 and 76 respectively. The spacing of buttons 74, 75 and 76 provides the above-mentioned size adjustment to accommodate patients. Belt 70 further includes a pair of front buttons 77 and 78. Buttons 77 and 78 are utilized in providing a button attachment to the attachment pads of an alternate embodiment of the present invention penis retractor. FIG. 16 sets forth an illustrative button hole bearing attachment pad. Suffice it to note here that buttons 74 through 76 cooperate with button hole 73 to secure abdominal belt 70 upon the patient while buttons 77 and 78 cooperate with the corresponding button holes such as button hole 102 of pad 101 (seen in FIG. 16) to secure the penis retractor.

FIG. 13 sets forth a further alternate embodiment of the abdominal belt cooperating with the present invention penis retractor which is substantially identical to belt 70 with the omission of front buttons 77 and 78. Thus, the present invention penis retractor in cooperation with belt 80 relies upon the above-described adhesive pad attachment for securing the attachment pads to the abdominal belt.

FIG. 14 sets forth a still further alternate embodiment of the cooperating abdominal belt portion of the present invention. Belt 90 defines an end 91 supporting a plurality of anchor tabs 94, 95, 96 and 97 while end 92 of belt 90 supports a cooperating hook 93. The cooperation of hook 93 within a selected one of anchor tabs 94 through 97 provides attachment of belt 90 to the user's abdomen. The spacing of anchor tabs 94 through 97 provides size adjustment.

FIG. 15 sets forth a further alternate embodiment of the cooperating abdominal belt utilized in the present invention penis retractor generally referenced by numeral 110. Belt 110 is substantially identical to belt 70 shown in FIG. 12 with the sole difference being found in the utilization of a single center button 79 in place of buttons 77 and 78. It will be apparent to those skilled in the art that belt 70 shown in FIG. 12 is preferably utilized in combination with retractor 40 shown in FIG. 4 and thus employs two buttons 77 and 78. It will be equally apparent to those skilled in the art that belt 110 is preferably used in combination with retractor 10 shown in FIG. 1 and thus provides a single center button 79 for use with a cooperating attachment pad having a button hole for attachment.

FIG. 16 sets forth a partial perspective view of an illustrative button hole attachment pad utilized in accordance with the present invention. Thus, in the example shown in FIG. 16, a tether 100 is joined to an attachment pad 101 having a button hole 102 therein. It will be apparent to those skilled in the art that in the embodiments of the present invention penis retractor employing a button attachment to a cooperating abdominal belt such as those shown in FIG. 12, 13 or 15, that the corresponding attachment pad or pads of the penis retractor which in other embodiments bare an adhesive layer will instead utilize a button hole attachment pad such as pad 101 shown in FIG. 16.

What has been shown is a secure yet comfortable penis retractor for use in surgical or examination procedures in which the need arises for restraining and retracting the patient's penis. The apparatus and it method of use illustrate a very comfortable yet very secure and reliable attachment which may be provided with a minimum of effort and expense.

While particular embodiments of the invention have been shown and described, it will be obvious to those skilled in the art that changes and modifications may be made without departing from the invention in its broader aspects. Therefore, the aim in the appended claims is to cover all such changes and modifications as fall within the true spirit and scope of the invention.

That which is claimed is:

1. A penis retractor used in restraining the penis of a patient during surgical, examination or treatment procedures, said penis retractor comprising:
an elongated flexible tether having first and second ends and a loop formed therein;
at least one attachment pad joined to said first end; and
means for adjusting said loop to encircle a portion of a patient's penis,
said tether being drawn to retract and position a patient's penis and said attachment pad being securable to a patient's abdomen to maintain said position.

2. The penis retractor set forth in claim 1 wherein said means for adjusting said loop includes slip knot formed in said tether by knotting said second end.

3. The penis retractor set forth in claim 2 wherein said attachment pad includes an adhesive portion.

4. The penis retractor set forth in claim 3 further including an abdominal belt constructed to encircle a portion of a patient's torso and wherein said attachment pad is attached to said abdominal belt.

5. The penis retractor set forth in claim 1 wherein said tether further includes a clamp engaging said tether proximate said loop and wherein said at least one attachment pad includes first and second attachment pads joined to said first and second ends.

6. The penis retractor set forth in claim 4 further including an abdominal belt constructed to encircle a portion of a patient's torso and wherein said attachment pads are attached to said abdominal belt.
